(19) United States
(12) Patent Application Publication
(10) Pub. No.: US 2024/0423993 A1
(43) Pub. Date: Dec. 26, 2024

(54) AMORPHOUS FORM OF RESMETIROM AND PROCESS FOR PREPARATION THEREOF

(71) Applicant: Zydus Lifesciences Limited, Ahmedabad (IN)

(72) Inventors: Kumar Kamlesh SINGH, Ahmedabad (IN); Kuldeep Natwarlal JAIN, Ahmedabad (IN); Naitik Bharatbhai PATEL, Ahmedabad (IN); Keval Ramnikbhai BAMBHAROLIYA, Ahmedabad (IN)

(21) Appl. No.: 18/751,872

(22) Filed: Jun. 24, 2024

(30) Foreign Application Priority Data
Jun. 23, 2023 (IN) 202321041877

(51) Int. Cl. A61K 31/53; A61K 9/10; A61K 47/32

(52) U.S. Cl. CPC A61K 31/53; A61K 9/10; A61K 47/32

(57) ABSTRACT

The present invention relates to an amorphous form of resmetirom. In particular, the present invention relates to an amorphous solid dispersion of resmetirom. The present invention relates to processes for the preparation of amorphous form of resmetirom. The present invention also relates to processes for the preparation of amorphous solid dispersion of resmetirom.

### FIELD OF THE INVENTION

The present invention relates to an amorphous form of resmetirom. The present invention also relates to process for preparing amorphous form of resmetirom. In particular, the present invention relates to an amorphous solid dispersion of resmetirom and their process for preparation. The present invention also relates to a pharmaceutical composition comprising amorphous form of resmetirom. The present invention also relates to a pharmaceutical composition comprising amorphous solid dispersion of resmetirom.

### BACKGROUND AND THE PRIOR ART

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

Resmetirom is a thyroid hormone receptor (THR) β-selective agonist. It is used in the treatment of non-alcoholic steatohepatitis (NASH), non-alcoholic fatty liver disease (NAFLD) and associated dyslipidemias. Its chemical name is 2-[3,5-dichloro-4-[(6-oxo-5-propan-2-yl-1H-pyridazin-3-yl)oxy]phenyl]-3,5-dioxo-1,2,4-triazine-6-carbonitrile, has the following chemical structure of Formula (I)

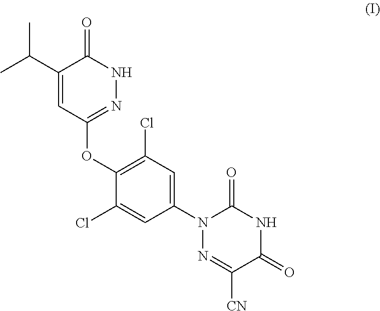 [SMILES: CC(C)c1cc(Oc2c(Cl)cc(-n3nc(C#N)c(=O)[nH]c3=O)cc2Cl)n[nH]c1=O]

U.S. Pat. No. 7,452,882 B2 discloses the compound resmetirom and provides its process for preparation.

U.S. Pat. No. 9,266,861 B2 discloses crystalline polymorph of resmetirom identified as Form I characterized by XRD.

U.S. Pat. No. 10,376,517 B2 discloses hydrates of resmetirom (monohydrate and dihydrate).

U.S. Pat. No. 11,564,926 B2 discloses resmetirom Form I (characterized by melting point & DSC peak), dimethylacetamide (DMAC) solvate of resmetirom, and a methyl isobutyl ketone (MBIK) solvate of resmetirom and method of treating resistance to thyroid hormone (RTH) syndrome.

U.S. Patent Appl. No. 2021/0122740 A1 discloses crystalline salt of resmetirom (wherein the counter-ion is selected from L-lysine, L-arginine, 2-hydroxy-N,N,N-trimethylethan-1-aminium, diethylamine, ethanolamine, ethanol-2-diethylamine, Na+, Mg2+, K+, Ca2+, diethanolamine, triethanolamine. L-histidine, and meglumine), solvated/desolvated polymorphic forms named as Forms B, C, D, E, F, G, H, I, K, L, S+T, S, U, V, W, X, Y, Z, alpha, beta, gamma, delta, epsilon, phi, eta, and lambda of resmetirom, a co-crystal of resmetirom and glutaric acid and amorphous solid dispersions of resmetirom.

PCT Pub. No. 2021063367 A1 relates to crystalline form CSI of resmetirom characterized by XRD.

U.S. Patent Appl. No. 20220372021 A1 relates to crystalline form CSIV of resmetirom characterized by XRD.

PCT Pub. No. 2022052822 A1 relates to crystalline form CSV1 of resmetirom characterized by XRD.

PCT Pub. No 2022171200 A1 relates to crystal form 3 of resmetirom characterized by XRD.

CN 115124515 A1 relates to crystalline forms named as Form 4, Form 7 and Form 9 of resmetirom characterized by XRD.

PCT Pub. No. 2022086894 A1 relates to crystalline solid-state forms of resmetirom: nicotinamide, resmetirom: caffeine, resmetirom: 2-picolinic acid, resmetirom: Urea, resmetirom N-methyl-morpholine salt, resmetirom piperazine salt, resmetirom benzathine salt and resmetirom: L-proline.

The prior-art discloses crystalline form of resmetirom. There is no disclosure of amorphous form of resmetirom.

In view of the above art, there is provided an amorphous form of resmetirom. In particular there is provided an amorphous form of resmetirom together with one or more pharmaceutically acceptable excipients and process for preparing thereof.

### SUMMARY OF THE INVENTION

In one general aspect, there is provided an amorphous form of resmetirom of Formula (I)

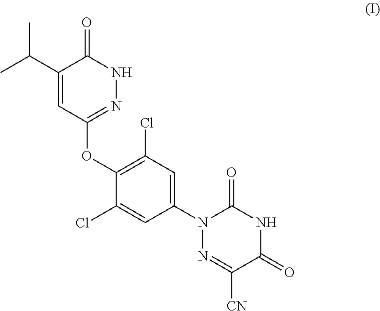 [SMILES: CC(C)c1cc(Oc2c(Cl)cc(-n3nc(C#N)c(=O)[nH]c3=O)cc2Cl)n[nH]c1=O]

In another general aspect, there is provided an amorphous solid dispersion of resmetirom of Formula (I)

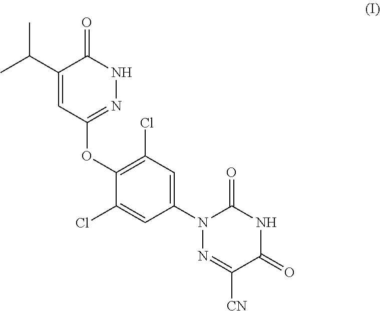 [SMILES: CC(C)c1cc(Oc2c(Cl)cc(-n3nc(C#N)c(=O)[nH]c3=O)cc2Cl)n[nH]c1=O]

together with one or more pharmaceutically acceptable excipients.

In another general aspect, there is provided a process for the preparation of an amorphous form of resmetirom using spray drying.

In another general aspect, there is provided a process for the preparation of an amorphous solid dispersion of resmetirom together with one or more pharmaceutically acceptable excipients, using spray drying.

In another general aspect, there is provided a process for the preparation of an amorphous form of resmetirom, the process comprising:
(a) providing a solution or suspension of resmetirom in the presence of solvent; and
(b) obtaining the amorphous form of resmetirom by the removal of the solvent.

In another general aspect, there is provided a process for the preparation of an amorphous solid dispersion of resmetirom together with one or more pharmaceutically acceptable excipients, comprising the steps of:
(a) providing a solution or suspension of resmetirom in the presence of one or more pharmaceutically acceptable excipients in one or more solvents; and
(b) obtaining the amorphous solid dispersion of resmetirom together with one or more pharmaceutically excipients by the removal of the solvents.

In another general aspect, there is provided a pharmaceutical composition comprising amorphous form of resmetirom together with one or more pharmaceutically acceptable excipients, carriers, or diluents.

In another general aspect, there is provided a pharmaceutical composition comprising amorphous form of resmetirom together with one or more pharmaceutically acceptable excipients, carriers, or diluents for the manufacture of medicaments for treating nonalcoholic steatohepatitis (NASH), non-alcoholic fatty liver disease (NAFLD) and or associated dyslipidemias.

In another general aspect, there is provided a pharmaceutical composition comprising amorphous solid dispersion of resmetirom together with one or more pharmaceutically acceptable excipients, carriers, or diluents.

### BRIEF DESCRIPTION OF THE ACCOMPANYING DRAWINGS

FIG. 1: X-ray powder diffractogram (XRPD) of amorphous form of resmetirom as per Example-1 and Example-2.

FIG. 2: X-ray powder diffractogram (XRPD) of amorphous solid dispersion of resmetirom with copovidone as per Example-3 and Example-4.

### DETAILED DESCRIPTION OF THE INVENTION

The aforementioned objectives of the present invention are fulfilled by one or more of the processes described herein.

All ranges recited herein include the endpoints, including those that recite a range “between” two values. Terms such as “about”, “from”, “generally”, “substantially,” and the like and “to” are to be construed as modifying a term or value such that it is not an absolute. This includes, at very least, the degree of expected experimental error, technique error and instrument error for a given technique used to measure a value.

As used herein, the term “solution” does not limit to a clear solution only and includes a hazy solution or slurry which is a heterogeneous mixture.

The term “pharmaceutical compositions” herein includes pharmaceutical formulations like tablets, pills, powders, liquids, suspensions, emulsions, granules, capsules, suppositories, or injection preparations.

The term “composition” used herein means a physical mixture of two or more components.

As used herein, the terms “obtaining” means isolating the amorphous form, amorphous solid dispersion of resmetirom by way of filtration, filtration under vacuum, centrifugation, and decantation. The product obtained may be further or additionally dried to achieve the desired moisture values. For example, the product may be dried in a tray drier, dried under vacuum and/or in a Fluid Bed Drier.

As used herein, the term “solid dispersion” means any solid composition having at least two components.

In certain embodiments, a solid dispersion as disclosed herein includes an active ingredient resmetirom thereof dispersed among at least one or more pharmaceutically acceptable excipients.

“Pharmaceutically acceptable” such as pharmaceutically acceptable excipient, carrier, or diluent, etc., means pharmacologically acceptable and substantially non-toxic to the subject to whom the particular compound is administered.

Suitable pharmaceutically acceptable excipients are not limited to diluents such as starches, pregelatinized starches, lactose, powdered celluloses, microcrystalline celluloses, dicalcium phosphate, tricalcium phosphate, polyethylene glycol, copovidone, soluplus, silicified microcrystalline cellulose mannitol, sorbitol, sugar and the like; binders such as acacia, guar gum, tragacanth, gelatin, polyvinylpyrrolidone (PVP), hydroxypropyl celluloses, hydroxypropyl methylcelluloses such as hydroxypropyl methylcellulose (HPMC), hydroxypropyl methylcellulose acetate succinate (HPMC-AS), hydroxypropyl methylcellulose phthalate (HPMCP), pregelatinized starches and the like; disintegrants such as starches, sodium starch glycolate, pregelatinized starches, copovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins and resins; release rate controlling agents such as hydroxypropyl celluloses, hydroxymethyl celluloses, hydroxypropyl methylcelluloses, ethylcelluloses, methylcelluloses, various grades of methyl methacrylates such as Eudragit L and Eudragit S, waxes and the like.

Other pharmaceutically acceptable excipients that are of use include but are not limited to film formers, plasticizers, colorants, flavoring agents, sweeteners, viscosity enhancers, preservatives, antioxidants, and the like.

Suitable pharmaceutically acceptable excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Such compositions will, in any event, contain an effective amount of the active compound together with a suitable carrier so as to prepare the proper dosage form for proper administration to the recipient.

The pharmaceutically acceptable excipients may also be selected from polymers. Polymers may be polyvinylpyrrolidone (PVP), 4-vinylpyrrolidone-vinyl acetate copolymer (copovidone), copolymers of methacrylic acid and ethylacrylate (EUDRAGIT® L100-55), hydroxypropyl cellulose, hydroxypropylmethyl cellulose (HPMC), hydroxypropyl methylcellulose phthalate (HPMCP), polymethyl acrylate, hypromellose phthalate, cellulose acetate phthalate and polymethacrylate or hydroxypropylmethyl cellulose acetate succinate (HPMC-AS).

Suitable pharmaceutically acceptable diluents are not limited to starches, pregelatinized starches, lactose, powdered celluloses, microcrystalline celluloses, dicalcium phosphate, and tricalcium phosphate. Common diluents include anhydrous lactose, lactose monohydrate, and sugar alcohols such as sorbitol, xylitol and mannitol. Diluents provide better tablet properties such as improved cohesion or to promote flow.

Useful pharmaceutically acceptable carriers for the preparation of the compositions thereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like.

The pharmaceutically acceptable carriers such as syloid, methyl cellulose, colloidal silicon dioxide, amorphous silica, micro crystalline cellulose, and the like has been found to be of particular value. Therefore, these ingredients may be combined during the preparation of pharmaceutical composition comprising amorphous resmetirom or amorphous solid dispersion of resmetirom to control hygroscopicity and to improve stability.

In one general aspect there is provided an amorphous form of resmetirom of Formula (I)

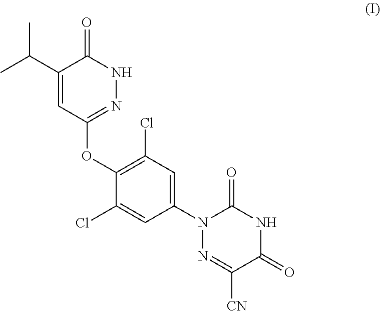 [SMILES: CC(C)c1cc(Oc2c(Cl)cc(-n3nc(C#N)c(=O)[nH]c3=O)cc2Cl)n[nH]c1=O]

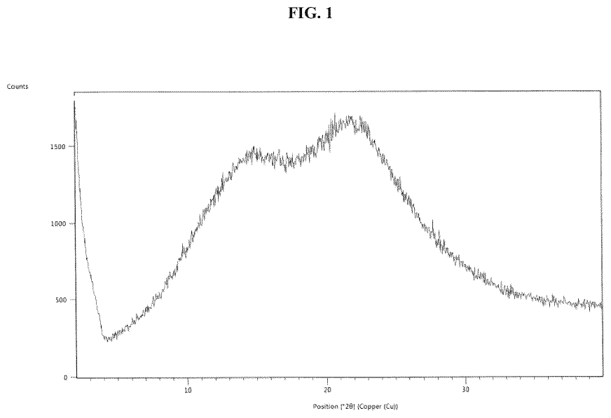

In another general aspect, there is provided an amorphous form of resmetirom, characterized by X-ray diffraction as depicted in FIG. 1.

In another general aspect, there is provided an amorphous form of resmetirom having purity of greater than about 99% by HPLC.

In another general aspect, there is provided amorphous form of resmetirom having water content from about 0.5% to about 5% wt/wt.

In another general aspect, there is provided an amorphous form of resmetirom substantially free from residual organic solvents.

In another general aspect there is provided an amorphous solid dispersion of resmetirom of Formula (I)

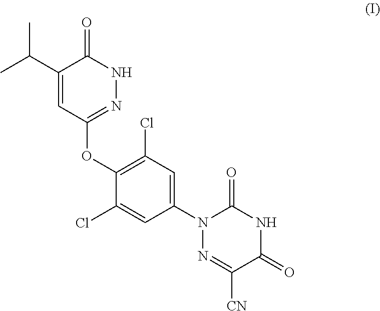 [SMILES: CC(C)c1cc(Oc2c(Cl)cc(-n3nc(C#N)c(=O)[nH]c3=O)cc2Cl)n[nH]c1=O]

together with one or more pharmaceutically acceptable excipients.

In another general aspect, there is provided an amorphous solid dispersion of resmetirom having purity of greater than about 99% by HPLC.

In another general aspect, there is provided amorphous solid dispersion of resmetirom having water content from about 0.5% to about 5% wt/wt.

In another general aspect, there is provided an amorphous solid dispersion of resmetirom substantially free from residual organic solvents.

In another general aspect, there is provided an amorphous solid dispersion of resmetirom with copovidone.

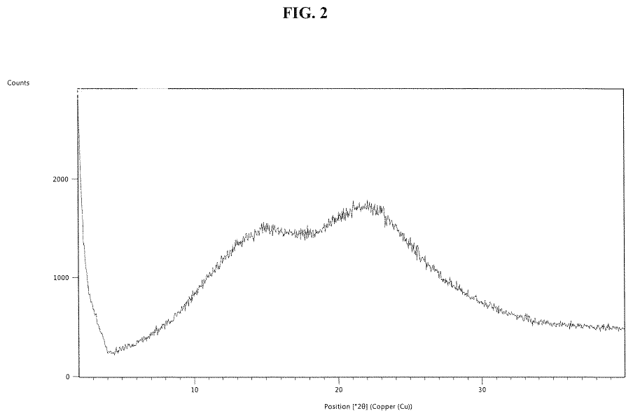

In another general aspect, there is provided an amorphous solid dispersion of resmetirom with copovidone, characterized by X-ray diffraction as depicted in FIG. 2.

In another general aspect, there is provided a process for preparation of an amorphous form of resmetirom, comprising the steps of:

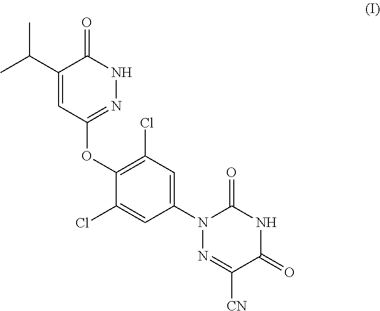 [SMILES: CC(C)c1cc(Oc2c(Cl)cc(-n3nc(C#N)c(=O)[nH]c3=O)cc2Cl)n[nH]c1=O]

(a) providing a solution of resmetirom in a solvent or mixture of solvents; and
(b) obtaining the amorphous form of resmetirom by removal of solvent.

In another general aspect, there is provided a process for preparation of an amorphous form of resmetirom, comprising the steps of:
(a) providing a solution of resmetirom in a solvent or mixture of solvents;
(b) adding an antisolvent to the solution obtained in step a); and
(c) isolating the amorphous form of resmetirom.

In another general aspect, there is provided a process for the preparation of an amorphous solid dispersion of resmetirom together with one or more pharmaceutically acceptable excipients, the process comprising:
(a) providing a solution or suspension of resmetirom and one or more pharmaceutically acceptable excipients in one or more solvents; and
(b) obtaining the amorphous solid dispersion of resmetirom together with one or more pharmaceutically acceptable excipients by removal of the solvents.

The step (a) above involves providing a solution or suspension of resmetirom in one or more of solvents or mixture thereof.

The solution or suspension for step (a) can be obtained by known methods that include:
(i) direct use of a reaction mixture containing resmetirom that is obtained in the course of its synthesis; or
(ii) dissolving resmetirom in one or more of solvents or mixture thereof.

In general, in step (a) any physical form of resmetirom may be utilized for providing the solution of resmetirom in one or more of solvents or mixture thereof. The dissolution temperatures may be from about below 0° C. to about the reflux temperature of the solvent.

In general, the solvent comprises one or more of C₁₋₄ alcohols, C₂₋₆ esters, ketones, halogenated hydrocarbons, polar aprotic solvents, tetrahydrofuran, 2-methyltetrahydrofuran, dioxane or mixtures thereof.

In general, the C₁₋₄ alcohol is selected from methanol, ethanol, n-propanol, isopropanol and n-butanol; the C₂₋₆ ester is selected from ethyl acetate, propyl acetate, isopropyl acetate, t-butyl acetate, and isobutyl acetate: the ketone is selected from acetone, methyl ethyl ketone, and methyl isobutyl ketone; the halogenated hydrocarbon is selected from methylene dichloride, ethylene dichloride, carbon tetrachloride and chlorobenzene; and the polar aprotic solvent is selected from dimethylformamide, dimethylsulfoxide, and N-methylpyrrolidone or mixture thereof.

In general, the pharmaceutically acceptable excipients may be selected from polymers. Polymers may be selected from methacrylic acid copolymers; polyvinylpyrrolidone (PVP), 4-vinylpyrrolidone-vinyl acetate copolymer (copovidone) or copolymers of methacrylic acid and ethylacrylate (EUDRAGIT® L100-55), hydroxy-propyl cellulose, hydroxypropylmethyl cellulose (HPMC), polymethylacrylate, hypromellose phthalate, or hydroxypropylmethyl cellulose acetate succinate (HPMC-AS). In particular. PVP of different grades like K-15, K-30, K-60. K-90 and K-120 may be used for the preparation of amorphous solid dispersion. More particular, hydroxypropylmethyl cellulose (HPMC) or hydroxypropylmethyl cellulose acetate succinate (HPMC-AS), 4-vinylpyrrolidone-vinyl acetate copolymer (copovidone) and PVP K-30 may be used.

In some embodiments, the ratio of the amount of weight of resmetirom within the solid dispersion to the amount by weight of the polymer therein is from about 1:1 to about 1:10. The composition of resmetirom with polymer may be prepared by using about 1:1 to about 1:10 polymers with respect to resmetirom.

In some embodiments, the ratio of the amount of weight of resmetirom and the amount by weight of the excipient is from about 1:1 to about 1:10.

The solution of resmetirom in presence of excipient in one or more solvents, preferably PVP K-30 or HPMC-AS may be prepared by using about 1:1 to about 1:10 polymers with respect to resmetirom.

The step (b) above involves obtaining of an amorphous solid dispersion of resmetirom from the solution or suspension of step (a). The isolation of an amorphous solid dispersion of resmetirom may be affected by removing the solvents. The techniques which may be used for the removal of solvents comprises one or more of distillation, distillation under vacuum, spray drying, agitated thin film drying (“ATFD”), freeze drying (lyophilization), filtration, decantation, and centrifugation. The solvent may also be removed, optionally, at reduced pressure and/or at elevated temperature.

Alternatively, isolation can be effected by addition of suitable antisolvent to the solution obtain in step a), optionally by concentrating the solution obtain in step a).

Suitable anti-solvents comprises of water, hydrocarbons selected from hexane, n-heptane, n-pentane, cyclohexane, methylcyclohexane; aromatic hydrocarbons selected from toluene, xylene, chlorobenzene, ethylbenzene and ethers selected from diethyl ether, diisopropyl ether, t-butyl methyl ether and dibutyl ether.

According to further general aspect, resmetirom may be spray dried by dissolving or suspending or slurring in suitable solvent to get amorphous form or amorphous solid dispersion of resmetirom.

In the present invention feed stock of resmetirom in solvent is spray-dried. Thus obtain spry-dried compound is in amorphous form or amorphous solid dispersion, confirmed by the x-ray powder diffractogram of spray-dried resmetirom In a specific preferred aspect of the invention, weighed quantity of resmetirom is dissolved in 2-10 volumes of chosen solvent, preferably 4-5 volumes solvent at 25° C. to 30° C. The content is stirred for 30 minutes at 25° C. to 30° C. The content is filtered through Hyflosupercell, and filtrate is spray dried under following conditions. The obtained powder is further dried at 40° C. for 12-16 hours under vacuum to afford amorphous form or amorphous solid dispersion of resmetirom.

Spray Dryer Parameter:

| Parameter | Probable Range | |
|---|---|---|
| Inlet Temperature | 70 to 80° | C. |
| Outlet Temperature | >50° | C. |
| Aspirator Flow rate | 50 to 110 | Nm³/hr |
| Vacuum | >−30 | mm/Hg |
| Feed Pump Flow rate | 3 to 4 | ml/min |

| | |
|---|---|
| Oxygen level | Below 5.0% |

In another general aspect, the present invention provides an amorphous form of resmetirom having purity of greater than 99% by HPLC. In particular, the purity of greater than 99.5% by HPLC, more particularly, the purity of greater than 99.8% by HPLC, most particularly, the purity of greater than 99.9% by HPLC.

In another general aspect, the present invention provides an amorphous solid dispersion of resmetirom with one or more pharmaceutically acceptable excipients, having purity by HPLC of greater than 99% by HPLC. In particular, the purity of greater than 99.5% by HPLC, more particularly, the purity of greater than 99.8% by HPLC, most particularly, the purity of greater than 99.9% by HPLC.

The invention also encompasses a pharmaceutical composition containing an amorphous form of resmetirom. As used herein, the term “pharmaceutical compositions” includes pharmaceutical formulations comprises one or more of tablets, pills, powders, liquids, suspensions, emulsions, granules, capsules, suppositories, and injection preparations.

In another general aspect, there is provided a pharmaceutical composition comprising amorphous form of resmetirom together with one or more pharmaceutically acceptable excipients, carriers, or diluents.

In another general aspect, there is provided a pharmaceutical composition comprising amorphous form of resmetirom together with one or more pharmaceutically acceptable excipients, carriers, or diluents for the manufacture of medicaments for treating nonalcoholic steatohepatitis (NASH), non-alcoholic fatty liver disease (NAFLD) and or associated dyslipidemias.

In another general aspect, there is provided a method of treating non-alcoholic steatohepatitis (NASH), non-alcoholic fatty liver disease (NAFLD) and or associated dyslipidemias by administering amorphous form of resmetirom together with one or more pharmaceutically acceptable excipients, carriers, or diluents.

In another general aspect, there is provided a pharmaceutical composition comprising an amorphous solid dispersion of resmetirom together with one or more pharmaceutically acceptable excipients, carriers, or diluents.

In general, the pharmaceutical compositions containing the amorphous solid dispersion of resmetirom with one or more pharmaceutically acceptable excipients, of the invention may be prepared by using diluents or excipients selected from fillers, bulking agents, binders, wetting agents, disintegrating agents, surface active agents and lubricants.

In another general aspect, there is provided a pharmaceutical composition comprising amorphous solid dispersion of resmetirom with copovidone and one or more pharmaceutically acceptable excipients, carriers, or diluents.

In another general aspect, there is provided a pharmaceutical composition comprising amorphous solid dispersion of resmetirom together with copovidone and one or more pharmaceutically acceptable excipients, carriers, or diluents for the manufacture of medicaments for treating nonalcoholic steatohepatitis (NASH), non-alcoholic fatty liver disease (NAFLD) and or associated dyslipidemias.

In another general aspect, there is provided a pharmaceutical composition comprising amorphous solid dispersion of resmetirom together with one or more pharmaceutically acceptable excipients, carriers, or diluents.

In another general aspect, there is provided a pharmaceutical composition comprising amorphous solid dispersion of resmetirom together with one or more pharmaceutically acceptable excipient, carriers, or diluents for the manufacture of medicaments for treating nonalcoholic steatohepatitis (NASH), non-alcoholic fatty liver disease (NAFLD) and or associated dyslipidemias.

In another general aspect, there is provided a method of treating non-alcoholic steatohepatitis (NASH), non-alcoholic fatty liver disease (NAFLD) and or associated dyslipidemias by administering amorphous solid dispersion of resmetirom together with one or more pharmaceutically acceptable excipients, carriers, or diluents.

The present invention is further illustrated by following examples which are provided merely to exemplify the invention and do not limit the scope of it.

### EXAMPLES

### Example-1: Preparation of Amorphous Form of Resmetirom

Spray Dryer Parameter:

| Parameter | Probable Range | |
|---|---|---|
| Inlet Temperature | 80 to 90° | C. |
| Outlet Temperature | >50° | C. |
| Aspirator Flow rate | 50 to 110 | Nm³/hr |
| Vacuum | >−30 | mm/Hg |
| Feed Pump Flow rate | 3 to 4 | ml/min |

| | |
|---|---|
| Oxygen level | Below 5.0% |

Methanol (3000 ml) and resmetirom (100 g) were added in RB flask at 25° C. to 35° C. and reaction mass was stirred for 15 to 20 minutes to get clear solution. The reaction mass was assembled in spray dryer. The reaction mass was spray dried under below conditions. The product was collected from cyclone and was further dried at 40° C. to 50° C. under vacuum for 4 hours to get amorphous form of resmetirom.

HPLC Purity: 99.62%.

### Example-2: Preparation of Amorphous Form of Resmetirom

Methanol (300 ml) and resmetirom (100 g) were added at 25° C. to 35° C. The reaction mass was stirred for 10 to 15 minutes to get a clear solution. Methanol was distilled out under vacuum below 45° C. from above reaction mass, n-Heptane (500 ml) was added to residue at 25° C. to 35° C. The reaction mass was stirred for 15 to 20 minutes, filtered and washed with n-Heptane (100 ml) to get amorphous form of resmetirom.

HPLC Purity: 99.6%.

### Example-3: Preparation of Amorphous Solid Dispersion of Resmetirom

Spray Dryer Parameter:

| Parameter | Probable Range | |
|---|---|---|
| Inlet Temperature | 70 to 80° | C. |
| Outlet Temperature | >50° | C. |
| Aspirator Flow rate | 50 to 110 | Nm³/hr |
| Vacuum | >−30 | mm/Hg |
| Feed Pump Flow rate | 3 to 4 | ml/min |

| | |
|---|---|
| Oxygen level | Below 5.0% |

Methanol (3000 ml), resmetirom (100 g) and copovidone (Kollidone VA 64) (100 g) were added in RB flask at 25° C. to 35° C. and reaction mass was stirred for 15 to 20 minutes to get clear solution. The reaction mass was assembled in spray dryer. The reaction mass was spray dried under below conditions. The product was collected from cyclone and was further dried at 40° C. to 50° C. under vacuum for 4 hours to get amorphous solid dispersion of resmetirom.

HPLC Purity: 99.62%.

### Example-4: Preparation of Amorphous Solid Dispersion Resmetirom

Methanol (3000 ml) and resmetirom (100 g) were added at 25° C. to 35° C. The reaction mass was stirred for 15 to 20 minutes to get a clear solution, copovidone (Kollidone VA 64) (100 g) was added to the reaction mass and stirred for 15 to 20 minutes and after that reaction mass was filtered out. Methanol was distilled out below 50° C. from above reaction mass. Cyclohexane (400 ml) was added to residue at 25° C. to 35° C. The reaction mass was stirred for 15 to 20 minutes, filtered and washed with cyclohexane (100 ml) to get amorphous solid dispersion of resmetirom.

HPLC Purity: 99.6%

We claim:

1. An amorphous form of resmetirom.

2. The amorphous form of resmetirom according to claim 1 characterized by X-ray diffraction as depicted in FIG. 1.

3. The amorphous form of resmetirom according to claim 1 having water content from about 0.5% to about 5% wt/wt.

4. The amorphous form of resmetirom according to claim 1 having purity of greater than about 99% by HPLC.

5. A process for the preparation of an amorphous form of resmetirom of claim 1 comprising the steps of:
(a) providing a solution of resmetirom in a solvent or mixture of solvents; and
(b) isolating the amorphous form of resmetirom by the removal of solvents.

6. The process according to claim 5, wherein the solvent may be selected from C₁₋₄ alcohols, C₂₋₆ esters, ketones, halogenated hydrocarbons, polar aprotic solvents, tetrahydrofuran, 2-methyltetrahydrofuran, dioxane, or mixtures thereof.

7. The process according to claim 5, wherein the removal of solvent comprises one or more of distillation, distillation under vacuum, spray drying, agitated thin film drying (“ATFD”), freeze drying (lyophilization), filtration, decantation, and centrifugation.

8. A process for the preparation of an amorphous form of resmetirom of claim 1 comprising the steps of:
(a) providing a solution of resmetirom in a solvent or mixture of solvents;
(b) adding an antisolvent to the solution obtained in step a); and
(c) isolating the amorphous form of resmetirom.

9. The process according to claim 8, wherein the solvent may be selected from C₁₋₄ alcohols, C₂₋₆ esters, ketones, halogenated hydrocarbons, polar aprotic solvents, tetrahydrofuran, 2-methyltetrahydrofuran, dioxane, or mixtures thereof.

10. The process according to claim 8, wherein the antisolvent may be selected from hexane, n-heptane, n-pentane, cyclohexane, methylcyclohexane, toluene, xylene, chlorobenzene, ethylbenzene, diethyl ether, diisopropyl ether, t-butyl methyl ether, and dibutyl ether.

11. An amorphous solid dispersion of resmetirom together with at least one pharmaceutically acceptable excipient.

12. The amorphous solid dispersion of resmetirom of claim 11, with copovidone.

13. The amorphous solid dispersion of resmetirom with copovidone according to claim 12, characterized by X-ray diffraction as depicted in FIG. 2.

14. A process for the preparation of an amorphous solid dispersion of resmetirom together with at least one pharmaceutically acceptable excipient, according to claim 11, comprising the steps of:
(a) providing a solution or suspension of resmetirom in the presence of at least one pharmaceutically acceptable excipient in one or more solvents; and
(b) obtaining the amorphous solid dispersion of resmetirom together with at least one pharmaceutically acceptable excipient by the removal of the one or more solvents.

15. The process according to claim 14, wherein the pharmaceutically acceptable excipient is selected from the group consisting of polyvinylpyrrolidone (PVP), 4-vinylpyrrolidone-vinyl acetate copolymer (copovidone), copolymers of methacrylic acid and ethylacrylate (EUDRAGIT® L100-55), hydroxypropyl cellulose, hydroxypropylmethyl cellulose (HPMC), polymethyl acrylate, hypromellose phthalate, or hydroxypropylmethyl cellulose acetate succinate (HPMC-AS).

16. The process according to claim 14, wherein the solvent may be selected from C₁₋₄ alcohols, C₂₋₆ esters, ketones, halogenated hydrocarbons, polar aprotic solvents, tetrahydrofuran, 2-methyltetrahydrofuran, dioxane, or mixtures thereof.

17. A pharmaceutical composition comprising an amorphous form of resmetirom together with one or more pharmaceutically acceptable excipients, carriers, or diluents.

18. The pharmaceutical composition comprising the amorphous form of resmetirom together with one or more pharmaceutically acceptable excipients, carriers, or diluents, according to claim 17, for the manufacture of medicaments for treating nonalcoholic steatohepatitis (NASH), non-alcoholic fatty liver disease (NAFLD) and or associated dyslipidemias.

19. A pharmaceutical composition comprising amorphous solid dispersion of resmetirom together with copovidone and one or more pharmaceutically acceptable excipients, carriers, or diluents.

20. A pharmaceutical composition comprising amorphous solid dispersion of resmetirom together with copovidone and one or more pharmaceutically acceptable excipients, carriers, or diluents, according to claim 19, for the manufacture of medicaments for treating nonalcoholic steatohepatitis (NASH), non-alcoholic fatty liver disease (NAFLD) and or associated dyslipidemias.
